(19) United States
(12) Patent Application Publication
(10) Pub. No.: US 2026/0013027 A1
(43) Pub. Date: Jan. 8, 2026

(54) PLASMA ELECTRODE STRUCTURE AND PLASMA DEVICE FOR SKIN SURFACE TREATMENT

(71) Applicant: Stalwart Elite Technology Co., Ltd., Taipei City (TW)

(72) Inventors: FEI-WEN HUANG, Taipei (TW); CHIH-TUNG LIU, Taipei (TW); CHUN-PING HSIAO, Taipei (TW); CHUN-HAO CHANG, Taipei (TW); YU-TING LIN, Taipei (TW)

(21) Appl. No.: 18/957,323

(22) Filed: Nov. 22, 2024

(30) Foreign Application Priority Data
Jul. 8, 2024 (TW) 113125517

(51) Int. Cl. H05H 1/24; A61N 1/44

(52) U.S. Cl. CPC H05H 1/2418; A61N 1/44; H05H 2245/34

(57) ABSTRACT

A plasma electrode structure for skin surface treatment includes a discharge electrode, a voltage-resistant dielectric layer covering the discharge electrode, and a buffer dielectric layer laminated to the voltage-resistant dielectric layer. The buffer dielectric layer is disposed at least between the voltage-resistant dielectric layer and a skin surface, and the buffer dielectric layer has a lower dielectric strength than the voltage-resistant dielectric layer.

### CROSS-REFERENCE TO RELATED APPLICATION

This application claims the priority benefit of Taiwan application serial no. 113125517, filed Jul. 8, 2024. The entirety of the above-mentioned patent application is hereby incorporated by reference herein and made a part of this specification.

### BACKGROUND

### Field of the Invention

The invention relates to a plasma electrode structure and a plasma device for skin surface treatment.

### Description of the Related Art

Currently, it is common to see the application of cold plasma generated at room temperature in skin beauty or treatment technologies. For example, cold plasma can accelerate wound healing or help skin recover faster after beauty treatments by stimulating skin cell regeneration and promoting blood circulation. Cold plasma also promotes cell division and metabolism, increasing skin elasticity and firmness, thus achieving anti-aging effects. It can also open the skin's pores and improve the absorption efficiency of skincare products or medications, thus further enhancing skin texture, appearance, and the therapeutic effect on the skin.

However, current plasma skin treatment devices often suffer from the problem of uneven discharge intensity, leading to localized high-density currents that cause users to experience stinging or burning sensations. Additionally, these devices typically use ambient air to generate ionized gas, making it crucial to design them in a way that maintains an appropriate air gap between the plasma skin treatment device and the skin surface.

### BRIEF SUMMARY OF THE INVENTION

In order to achieve one or a portion of or all of the objects or other objects, one embodiment of the invention provides a plasma electrode structure for skin surface treatment including a discharge electrode, a voltage-resistant dielectric layer covering the discharge electrode, and a buffer dielectric layer laminated to the voltage-resistant dielectric layer. The buffer dielectric layer is disposed at least between the voltage-resistant dielectric layer and a skin surface, and the buffer dielectric layer has a lower dielectric strength than the voltage-resistant dielectric layer.

Another embodiment of the invention provides a plasma device for skin surface treatment including a power circuit, a transformer circuit configured to convert an output signal of the power circuit into a high-voltage signal, and a plasma electrode structure configured to receive the high-voltage signal to ionize gas and generate plasma acting on a skin surface. The plasma electrode structure includes a discharge electrode, a first dielectric layer and a second dielectric layer. The first dielectric layer covers the discharge electrode, the second dielectric layer disposed at least on a side of the first dielectric layer facing the skin surface, the second dielectric layer is made of a different material from the first dielectric layer, and the second dielectric layer has a lower dielectric constant than the first dielectric layer.

Through the design of the above embodiments, by using the buffer dielectric layer with a lower dielectric strength than the voltage-resistant dielectric layer, the discharge intensity and uniformity can be adjusted to prevent localized high current density, thus ensuring that the user does not experience discomfort or burning sensations on the skin. Besides, the design of the above embodiments can accommodate various types drive sources with different waveforms to increase flexibility in choosing the appropriate type of drive circuit and to simplify the design process for the drive circuit. Furthermore, the design of a spacer around the periphery of a plasma electrode structure can create an air chamber on the skin surface, ensuring sufficient gas to generate plasma. This design also helps maintain a proper distance from the skin surface, leading to uniformly distributed plasma that effectively targets the desired skin area.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 shows a schematic diagram of a plasma device for skin surface treatment according to an embodiment of the invention.

FIG. 2 shows a schematic diagram of a plasma electrode structure according to an embodiment of the invention.

FIG. 3 shows a schematic diagram of a plasma electrode structure according to another embodiment of the invention.

FIG. 4 shows a schematic diagram of a multi-layer discharge electrode according to an embodiment of the invention.

FIG. 5 shows a schematic diagram of a plasma electrode structure according to another embodiment of the invention.

FIG. 6 shows actual effects of a conventional single-layer dielectric design, and FIG. 7 shows actual effects of a dual-layer dielectric design according to various embodiments of the invention.

### DETAILED DESCRIPTION OF THE INVENTION

In the following detailed description of the preferred embodiments, directional terminology, such as “top,” “bottom,” “front,” “back,” etc., is used with reference to the orientation of the Figure(s) being described. The components of the invention can be positioned in a number of different orientations. As such, the directional terminology is used for purposes of illustration and is in no way limiting. Further, “First,” “Second,” etc, as used herein, are used as labels for nouns that they precede, and do not imply any type of ordering (e.g., spatial, temporal, logical, etc.).

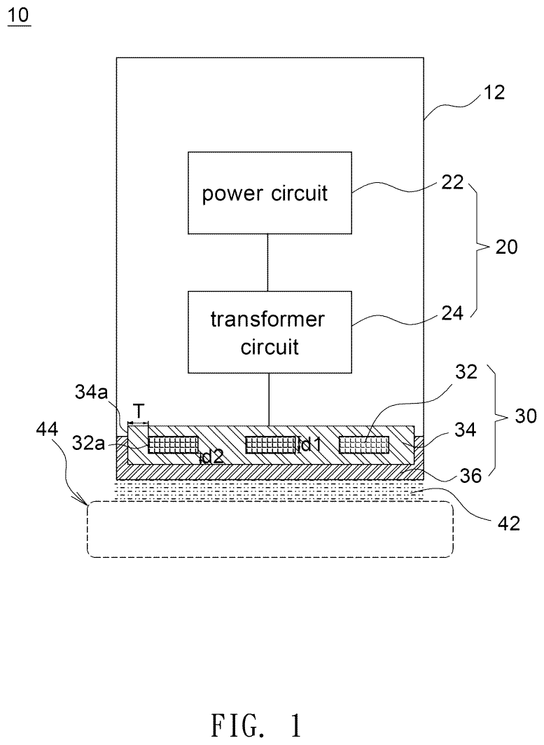

FIG. 1 shows a schematic diagram of a plasma device for skin surface treatment according to an embodiment of the invention. As shown in FIG. 1, a plasma device 10 includes a drive unit 20 and a plasma electrode structure 30 disposed within a housing 12. In this embodiment, the drive unit 20 includes a power circuit 22 and a transformer circuit 24. The power circuit 22 may include a DC voltage source such as a battery and a high-frequency oscillator to generate a low-voltage high-frequency signal. The transformer circuit 24 can boost the low-voltage high-frequency signal from the power circuit 22 to the required high voltage level, converting it into a high-voltage signal. The high-voltage signal is transmitted to the plasma electrode structure 30 to ionize gas to generate plasma 42. The plasma 42 can act on the skin surface 44 for various cosmetic or therapeutic treatments. In one embodiment, the signal frequency input to the plasma electrode structure 30 can be greater than 20 kHz to avoid generating noise audible to the human ear, and the transformer circuit 24 may have an intermittent power supply function to adjust the average intensity of the plasma.

In this embodiment, the plasma electrode structure 30 includes at least one discharge electrode 32, a voltage-resistant dielectric layer 34, and a buffer dielectric layer 36. As shown in FIG. 1, the voltage-resistant dielectric layer 34 covers the discharge electrode 32, and the buffer dielectric layer 36 is laminated to the voltage-resistant dielectric layer 34 and disposed between the voltage-resistant dielectric layer 34 and the skin surface 44. Note that the term “covers” only means the voltage-resistant dielectric layer 34 is allowed to cover the main area of the discharge electrode 32 to prevent electric arc discharge caused by excessive current, and does not imply that the voltage-resistant dielectric layer 34 must cover all surface areas of the discharge electrode 32. In one embodiment, as shown in FIG. 1, the outer surface of the discharge electrode 32 can be completely covered by the voltage-resistant dielectric layer 34, and the shortest distance T from a side surface 32a of the discharge electrode 32 to a side surface 34a of the voltage-resistant dielectric layer 34 is not less than 0.5 mm. In this embodiment, the buffer dielectric layer 36 is made of a different material from the voltage-resistant dielectric layer 34. Since the voltage-resistant dielectric layer 34 covers the high-voltage discharge electrode 32, the material of the voltage-resistant dielectric layer 34 needs to withstand high temperatures and be resistant to arc breakdown. Therefore, the voltage-resistant dielectric layer 34 is made of materials with relatively high dielectric constant and dielectric strength, such as quartz, ceramics, glass, or a composite material containing at least one of quartz, ceramics and glass. The buffer dielectric layer 36 can provide additional protection for the skin surface 44, and can be used to adjust the discharge intensity and uniformity, thus preventing localized high current density and ensuring that the user does not experience discomfort or burning sensations due to high-temperature current surges. In this embodiment, the buffer dielectric layer 36 can be made of materials with relatively low dielectric constant and dielectric strength as compared with the voltage-resistant dielectric layer 34, such as Teflon, plastic, silicone, or a composite containing at least one of Teflon, plastic and silicone. In one embodiment, the dielectric constant of the voltage-resistant dielectric layer 34 may range from 5 to 15, the dielectric strength of the voltage-resistant dielectric layer 34 may range from 10 to 300 kV/mm, the dielectric constant of the buffer dielectric layer 36 may range from 1 to 5, and the dielectric strength of the buffer dielectric layer 36 may range from 10 to 60 kV/mm. In one embodiment, a printed circuit electrode layer may be formed on stacked dielectric thin layers and then covered with another dielectric thin layer. The assembly is subjected to high temperatures for sintering and pressing, causing the dielectric materials to fuse and encapsulate the metal electrode layer. This process forms the discharge electrode 32 and the voltage-resistant dielectric layer 34. However, the invention is not limited to this specific method. The discharge electrode 32 can be made of metal materials, such as aluminum, magnesium, titanium, silver, copper, iron, or their alloys.

In one embodiment, as shown in FIG. 1, a thickness d1 of the discharge electrode 32 is preferably less than 10 μm to improve the flatness of the discharge end surface, and a thickness d2 of the voltage-resistant dielectric layer 34 is within the range of 200 μm<d2<5000 μm, more preferably within the range of 200 μm<d2<2000 μm. The thickness d2 meeting the condition of greater than 200 μm ensures the generation of plasma and prevents the electric arcs from breaking down the voltage-resistant dielectric layer 34. Besides, the thickness d2 meeting the condition of less than 5000 μm may avoid cost inefficiencies due to excessive thickness. Herein, the thickness d2 of the voltage-resistant dielectric layer 34 is defined as the shortest distance measured from the discharge electrode 32 to the lower surface of the voltage-resistant dielectric layer 34 (i.e., a surface of the voltage-resistant dielectric layer 34 adjoining the buffer dielectric layer 36).

In one embodiment, a thickness of the buffer dielectric layer 36 may range from 10 to 200 μm. Generally, the shorter the rise time of the drive waveform used to generate plasma, the more it can avoid excessive current during plasma discharge. Herein, the term “rise time” is defined as the time required for the signal to rise from a low level (10% level) to a high level (90% level). When the rise time of the drive waveform is greater than 1500 ns, it is very likely to produce high-temperature current surges, causing discomfort or burning sensations on the skin surface. Therefore, the design that incorporates the buffer dielectric layer 36 in the above embodiment provides uniform discharge intensity to avoid localized high current density. This design ensures that even when a waveform with a rise time greater than 1500 ns (such as a sine wave) is used to drive the system, high-temperature current surges are still prevented. Thus, the design of the above embodiments can accommodate various types drive sources with different waveforms to increase flexibility in choosing the appropriate type of drive circuit and simplifying the design process for the drive circuit.

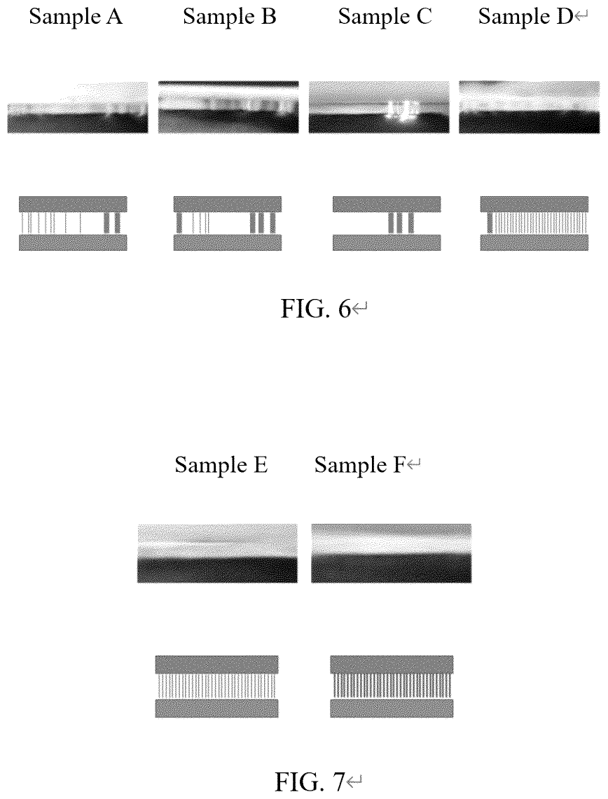

FIG. 6 and FIG. 7 show comparison diagrams of actual effects of different dielectric structure samples from conventional designs and the embodiments of the invention. In FIG. 6, samples A, B, C and D represent conventional single-layer dielectric designs, while samples E and F in FIG. 7 represent dual-layer dielectric designs according to various embodiments of the invention. The structure composition and layer thickness of samples A-F are shown in Table 1 below. The top rows in FIG. 6 and FIG. 7 display actual test photos illustrating plasma performance. The bottom rows in FIG. 6 and FIG. 7 provide schematic diagrams that represent the corresponding plasma intensity and distribution, depicted using line thickness and position. In FIG. 6, samples A, B, and C are electrode structures that each include only a voltage-resistant dielectric layer of varying thicknesses. As shown in FIG. 6, when only the voltage-resistant dielectric layer 34 is used, regardless of its thickness, plasma performance is highly uneven. This unevenness is due to the high dielectric constant of the voltage-resistant dielectric layer 34, which easily accumulates electric charge to thus lead to non-uniform plasma distribution and localized high current density. This can cause the electrode structure or the treated object to burn or become damaged, thus reducing its lifespan. If the treated object is human or skin, it can result in severe irritation (localized high heat) and even burns. Furthermore, sample D in FIG. 6 is an electrode structure with only the buffer dielectric layer 36. Although the plasma produced by sample D is relatively uniform, the lower voltage resistance of the buffer dielectric layer 36 may result in a very short lifespan, making it unsuitable for long-term use. Samples E and F in FIG. 7 represent embodiments of the invention where both the voltage-resistant dielectric layer 34 and the buffer dielectric layer 36 are used. The difference between samples E and F is the thickness of the voltage-resistant dielectric layer 34. As shown in FIG. 7, adding a buffer dielectric layer 36 outside the voltage-resistant dielectric layer 34 allows the plasma to be generated very uniformly without localized high current density, thus making it safe for use on human skin. This is because the low dielectric constant characteristic of the buffer dielectric layer 36 reduces charge accumulation and thus allows for uniform plasma distribution. FIG. 7 also demonstrates that, with a consistent thickness of the buffer dielectric layer 36 to achieve uniform plasma distribution, different plasma intensities can be obtained by adjusting the thickness of the voltage-resistant dielectric layer 34 to meet various practical needs.

TABLE 1

| | |
|---|---|
| Sample A | Only voltage-resistant dielectric layer 34 (thickness: 1 mm) |
| Sample B | Only voltage-resistant dielectric layer 34 (thickness: 0.38 mm) |
| Sample C | Only voltage-resistant dielectric layer 34 (thickness: 0.1 mm) |
| Sample D | Only buffer dielectric layer 36 (thickness: 0.1 mm) |
| Sample E | voltage-resistant dielectric layer 34 (thickness: 1 mm) |
| | plus buffer dielectric layer 36 (thickness: 0.1 mm) |
| Sample F | voltage-resistant dielectric layer 34 (thickness: 0.1 mm) |
| | plus buffer dielectric layer 36 (thickness: 0.1 mm) |

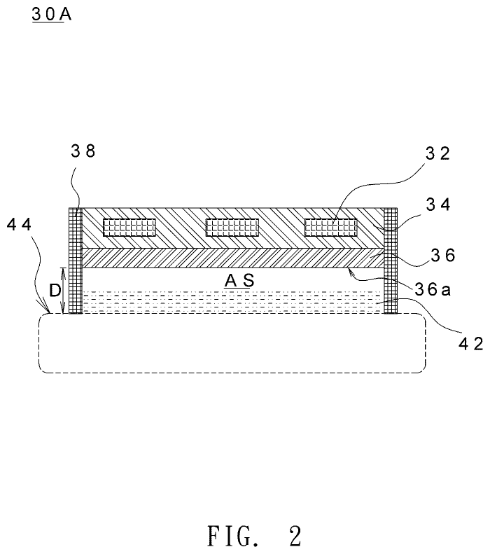

FIG. 2 shows a schematic diagram of a plasma electrode structure according to another embodiment of the invention. As shown in FIG. 2, a space between a discharge end (the bottom surface 36a of the buffer dielectric layer 36) of the plasma electrode structure 30A and the skin surface 44 is filled with air, and this space is occupied by plasma once generated. If the distance D is too short, there is insufficient air to generate adequate plasma 42. Conversely, if the distance is too long, it requires the accumulation of extremely high intensity charges to initiate plasma discharge, causing the discharge intensity at certain single point to become too strong. Therefore, in one embodiment, the distance D preferably ranges from 0.2 mm to 3 mm to avoid the above problem. Because the skin surface 44 that the discharge end of the plasma device touches might be elastic or curved, pressing during contact can squeeze out local gas. This can result in not having enough gas to generate plasma. Therefore, in this embodiment, a spacer 38, such as a cushion layer, can be provided around the periphery of the plasma electrode structure 30A and extended downward for a certain length, forming a reserved space for an air chamber AS between the plasma electrode structure 30A and the skin surface 44 to ensure sufficient gas to generate plasma. Besides, the spacer 38 may help maintain an appropriate distance from the skin surface 44 to generate uniformly distributed plasma that acts uniformly on the target area of the skin.

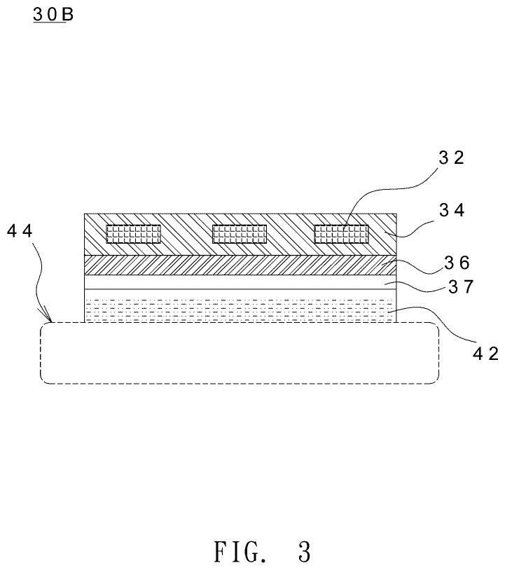

In various embodiments of the invention, the plasma electrode structure may have multiple dielectric layers made of different materials, and the number of dielectric layers is not limited. As shown in FIG. 3, the plasma electrode structure 30B includes not only a voltage-resistant dielectric layer 34 and a buffer dielectric layer 36 but also a third dielectric layer 37 made of a different material from the voltage-resistant dielectric layer 34 and the buffer dielectric layer 36. The dielectric layer 37 is provided on the side of the buffer dielectric layer 36 facing the skin surface 44. The provision of the dielectric layer 37 can further adjust the current intensity and uniformity, ensuring that the user's skin does not experience any discomfort. The material of the dielectric layer 37 can be selected as needed to provide additional effects. For example, the dielectric layer 37 can be made of a polymer resin material to provide both aesthetic and protective functions.

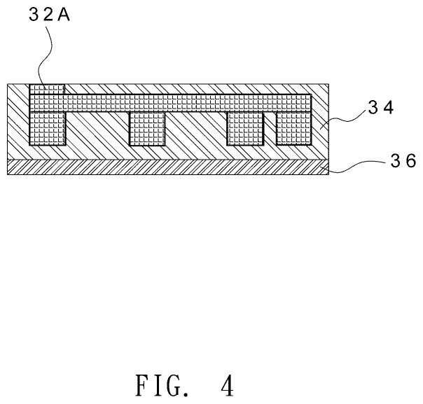

In accordance with various embodiments of the invention, the configuration of the discharge electrode is not restricted. For example, it may be a single-layer structure as shown in FIG. 1, or a multi-layer stacked structure as shown in FIG. 4. As shown in FIG. 4, the discharge electrode 32A featuring a multi-layer stacked structure (e.g., the illustrated three-layer structure) may enhance the flexibility to adjust plasma intensity and current density and may allow more diverse electrode pattern designs.

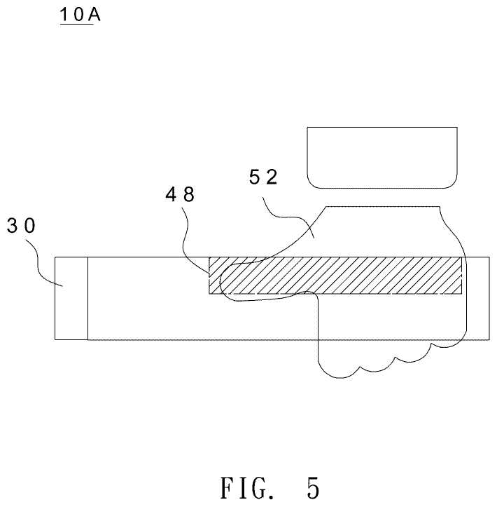

FIG. 5 illustrates a plasma device for skin surface treatment according to another embodiment of the invention. As shown in FIG. 5, the plasma device 10A can be configured as a handheld device, with a grounding electrode 48 provided at a position corresponding to the user's hand 52. When the user uses the plasma device 10A to treat the skin and the hand 52 contacts the grounding electrode 48, the plasma device 10A forms a discharge loop with the hand 52 to effectively control the discharge state of the plasma.

Through the design of the above embodiments, by using the buffer dielectric layer with a lower dielectric strength than the voltage-resistant dielectric layer, the discharge intensity and uniformity can be adjusted to prevent localized high current density, thus ensuring that the user does not experience discomfort or burning sensations on the skin. Besides, the design of the above embodiments can accommodate various types drive sources with different waveforms to increase flexibility in choosing the appropriate type of drive circuit and to simplify the design process for the drive circuit. Furthermore, the design of a spacer around the periphery of a plasma electrode structure can create an air chamber on the skin surface, ensuring sufficient gas to generate plasma. This design also helps maintain a proper distance from the skin surface, leading to uniformly distributed plasma that effectively targets the desired skin area.

Though the embodiments of the invention have been presented for purposes of illustration and description, they are not intended to be exhaustive or to limit the invention. Accordingly, many modifications and variations without departing from the spirit of the invention or essential characteristics thereof will be apparent to practitioners skilled in this art. It is intended that the scope of the invention be defined by the claims appended hereto and their equivalents in which all terms are meant in their broadest reasonable sense unless otherwise indicated.

What is claimed is:

1. A plasma electrode structure for skin surface treatment, comprising:
a discharge electrode;
a voltage-resistant dielectric layer covering the discharge electrode; and
a buffer dielectric layer laminated to the voltage-resistant dielectric layer and disposed at least between the voltage-resistant dielectric layer and a skin surface, wherein the buffer dielectric layer has a lower dielectric strength than the voltage-resistant dielectric layer.

2. The plasma electrode structure as claimed in claim 1, wherein the voltage-resistant dielectric layer has a dielectric strength ranging from 10 to 300 kV/mm, and the buffer dielectric layer has a dielectric strength ranging from 10 to 60 kV/mm.

3. The plasma electrode structure as claimed in claim 1, wherein the voltage-resistant dielectric layer has a dielectric constant ranging from 5 to 15, and the buffer dielectric layer has a dielectric constant ranging from 1 to 5.

4. The plasma electrode structure as claimed in claim 1, wherein the voltage-resistant dielectric layer is made of quartz, ceramics, glass, or a composite material containing at least one of quartz, ceramics and glass.

5. The plasma electrode structure as claimed in claim 1, wherein the buffer dielectric layer is made of Teflon, plastic, silicone, or a composite material containing at least one of Teflon, plastic and silicone.

6. The plasma electrode structure as claimed in claim 1, wherein an outer surface of the discharge electrode is completely covered by the voltage-resistant dielectric layer, and a shortest distance measured from a side surface of the discharge electrode to a side surface of the voltage-resistant dielectric layer is not less than 0.5 mm.

7. The plasma electrode structure as claimed in claim 1, wherein a thickness of the voltage-resistant dielectric layer ranges from 200 to 2000 μm.

8. The plasma electrode structure as claimed in claim 1, wherein a thickness of the buffer dielectric layer ranges from 10 to 200 μm.

9. The plasma electrode structure as claimed in claim 1, further comprising: a spacer defining an air chamber on the skin surface.

10. The plasma electrode structure as claimed in claim 1, wherein a thickness of the discharge electrode is less than 10 μm.

11. The plasma electrode structure as claimed in claim 1, further comprising:
a resin dielectric layer disposed on a side of the buffer dielectric layer facing the skin surface.

12. The plasma electrode structure as claimed in claim 1, wherein the discharge electrode is made from aluminum, magnesium, titanium, silver, copper, or iron.

13. A plasma device for skin surface treatment, comprising:
a power circuit;
a transformer circuit configured to convert an output signal of the power circuit into a high-voltage signal; and
a plasma electrode structure configured to receive the high-voltage signal to ionize gas and generate plasma acting on a skin surface, wherein the plasma electrode structure comprises:
a discharge electrode;
a first dielectric layer covering the discharge electrode; and
a second dielectric layer disposed at least on a side of the first dielectric layer facing the skin surface, the second dielectric layer being made of a different material from the first dielectric layer, and the second dielectric layer having a lower dielectric constant than the first dielectric layer.

14. The plasma device as claimed in claim 13, wherein a signal frequency input to the plasma electrode structure is greater than 20 kHz, and the transformer circuit has an intermittent power supply function.

15. The plasma device as claimed in claim 13, wherein the plasma device is configured as a handheld device, and the plasma device has a grounding electrode disposed at a position corresponding to a user's hand.

16. The plasma device as claimed in claim 13, wherein the first dielectric layer has a dielectric strength ranging from 10 to 300 kV/mm, and the second dielectric layer has a dielectric strength ranging from 10 to 60 kV/mm.

17. The plasma device as claimed in claim 13, wherein the first dielectric layer has a dielectric constant ranging from 5 to 15, and the second dielectric layer has a dielectric constant ranging from 1 to 5.

18. The plasma device as claimed in claim 13, wherein the first dielectric layer is made of quartz, ceramics, glass, or a composite material containing at least one of quartz, ceramics and glass.

19. The plasma device as claimed in claim 13, wherein the second dielectric layer is made of Teflon, plastic, silicone, or a composite material containing at least one of Teflon, plastic and silicone.

20. The plasma device as claimed in claim 13, further comprising:
a spacer defining an air chamber on the skin surface.
